(19) United States
(12) Patent Application Publication
(10) Pub. No.: US 2016/0278864 A1
(43) Pub. Date: Sep. 29, 2016

(54) Apparatus And Method For Instrument And Gesture Based Image Guided Surgery

(71) Applicant: Medtronic Navigation, Inc., Louisville, CO (US)

(72) Inventor: Yvan PAITEL, Louisville, CO (US)

(21) Appl. No.: 14/663,006

(22) Filed: Mar. 19, 2015

(51) Int. Cl. A61B 5/05

(52) U.S. Cl. CPC A61B 19/5244; A61B 2019/5236; A61B 2019/524

(57) ABSTRACT

A system is disclosed that allows for determining a position of an instrument in an object space. The position may include a three-dimensional location and at least one degree of freedom of orientation, or any appropriate number of degrees of freedom. The tracked position may be based on imaging the instrument, including imaging an external contour of the instrument. The movement of the instrument can be determined based on a three-dimensional determination of a movement of the instrument contour

### FIELD

The subject disclosure is related to determining a location of an instrument relative to an object, where the object may be a living or non-living object, and for displaying the location of the instrument relative to the object on a display device.

### BACKGROUND

This section provides background information related to the present disclosure which is not necessarily prior art.

In performing a procedure on an object, a user may often need to move an instrument relative to the object while the instrument is covered or internal to the object. Various imaging techniques can be used to obtain images of an internal portion of an object, but generally optical systems or human eyesight cannot see through an opaque exterior of the object. Objects can include humans, fuselages, mechanical systems (e.g., engines, condensers, and other systems) that include internal components that may require maintenance over time. It may be desirable, therefore, to have a system that allows for determining the location of an instrument relative to an internal component of the object based upon an image that is acquired with an imaging system that is able to image an internal portion of the object.

### SUMMARY

This section provides a general summary of the disclosure, and is not a comprehensive disclosure of its full scope or all of its features.

A system is disclosed that allows for determining the location of an instrument relative to an object space (which also may be referred to as patient space or subject space) which includes a three-dimensional location and three-dimensional orientation, or any appropriate number of dimensions, in real space. The position of an instrument can be tracked with a tracking localizer that allows for determining the location of the instrument within the object by tracking at least a portion of the instrument or a tracking device connected to the instrument. Further, the tracking system can be used to illustrate a projected line from the instrument into the object based upon the current tracked position of the object. Further, it is understood, that the position of the instrument can include both a location that can include a three-dimensional coordinate location of the instrument and an orientation that can include a six degree of freedom orientation at the tracked location. The combination of the location and the orientation may be referred to as a position of the instrument, which can be determined with the tracking system, as discussed further herein.

The tracking system may include a substantially portable localizer element system that may be selectively connected to a mounting or holding system that is associated with the object. By positioning the localizer relative to the object, the localizer can be used to track the position of the instrument relative to the object. When tracked, the position of the instrument may be displayed with a display device relative to an image of the object, including internal portions of the object. The localizer can be a relatively small and movable system to assist in determining a position of the instrument. The localizer may operate by various techniques such as an optical tracking technique that may use stereoscopic cameras or multiple camera lenses to image the instrument in space to allow for a determination of the position of the instrument.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a diagrammatic illustration of a system for determining a position of an instrument;

FIG. 2 is an exemplary use of a system for determining a position of an instrument;

FIG. 3 is a plan view of an instrument; and

FIG. 4 is a flowchart of use of a system for determining a position of an instrument.

Corresponding reference numerals indicate corresponding parts throughout the several views of the drawings.

### DETAILED DESCRIPTION

Example embodiments will now be described more fully with reference to the accompanying drawings.

A tracking system that may register image space (generally defined by an image that is displayed) to object space (generally the space defined in and around a selected object) can include a STEALTHSTATION® TRI®, TREON®, and/or S7™ Navigation System having an optical localizer, similar to the optical localizer 40, sold by Medtronic Navigation, Inc. having a place of business in Louisville, Colo. In various embodiments, object or subject space and image space can be registered by identifying matching points or fiducial points in the object space and related or identical points in the image space. When the position of an imaging device (not illustrated) is known, either through tracking or its “known” position (e.g. O-arm® imaging device sold by Medtronic, Inc.), or both relative to the object during imaging, the image data is generated at a precise and known position. This can allow image data that is automatically or “inherently registered” to the object being imaged upon acquisition of the image data.

Alternatively, manual or automatic registration can occur by matching fiducial points in image data with fiducial points on the object. Registration of image space to object space allows for the generation of a translation map between the object space and the image space. According to various embodiments, registration can occur by determining points that are substantially identical in the image space and the object space. The identical points can include anatomical fiducial points or implanted fiducial points. Exemplary registration techniques are disclosed in U.S. Pat. No. 8,842,893 and U.S. Pat. App. Pub. No. 2010/0228117, both incorporated herein by reference.

Once registered, a navigation system, as discussed herein, can be used to perform selected procedures. Selected procedures can use the image data generated or acquired with the imaging system. Further, the imaging system can be used to acquire image data at different times relative to a procedure. As discussed herein, image data can be acquired of the object subsequent to a selected portion of a procedure for various purposes, including confirmation of the portion of the procedure.

According to various embodiments, an object 20 can be placed in a three-dimensional space. The space contained within the object and a portion of space near the object may be referred to as an object space. The object 20 may define or include an interior volume 22 which may include an internal object or component 24. An opening or portal 26 may be provided or made in the object 20 into which an instrument 30 may be inserted. The instrument 30, which may be a stylus, a drill, an awl, etc., can be tracked relative to the object 20 with a localizer system 40. The localizer system 40 can include various components including a first lens 42 and a second lens 44. Both of the lenses 42, 44 may be connected to a single camera or each of the lenses may be part of separate cameras, therefore two separate cameras may be included in the localizer 40. Additionally, illumination structures or the like 46 can be provided that may illuminate the instrument 30 to assist in allowing the lenses 42, 44 to capture an image of the instrument 30.

The lenses 42, 44 can image the instrument 30 and the objects 20 to determine a relative position. The two lenses 42, 44 may view and be used to determine a depth or three-dimensional image or position of an instrument using stereoscopic techniques, generally understood in the art. Alternatively, as is generally understood in the art as discussed above, registration may occur between the object 20 in object space and an image of the object, including the internal object portion 24. An image can include an image 60 illustrated on a display device 66 which may include a monitor screen of a system 70, such as a computer system including a laptop computer, tablet computer, or the like.

The computer system may be a navigation computer 70 including at least a processor system 70a and a memory 70b. Both the memory system 70b and the processor system 70a may be incorporated with the computer system or be accessed by the system 70. Further, the processor 70a may be a general purpose processor that executes instructions in the form of code to complete selected tasks. The processor 70a may alternatively be an application specific integrated circuit (ASIC) or include portions that are application specific. The memory system 70b may be any appropriate type of memory such as a solid state, random access, removable disk, or the like.

The display 66 may also display an icon 30′ that illustrates the tracked position of the instrument 30′ relative to the object 20 as a position of the instrument icon 30′ relative to the object, including the internal object 24. The icon 30′ of the instrument may substantially appear as the instrument on the display. Further, the icon 30′ may include a three-dimensional rendering of the instrument 30. Accordingly, a rendering or an icon 24′ can be illustrated in the image 60. Further, the icon 30′ of the instrument can be superimposed on the object image 24′ when it is determined that the instrument 30 is over or in contact with the object 24. Further, a projected line, path or trajectory of the instrument 30 can be illustrated and superimposed on the object 24′ as well.

In various embodiments, the localizer system 40 can include a motion tracking device that can track the instrument 30 or other appropriate portions without a specific tracking member affixed thereto. Various systems can include the LEAP MOTION® motion sensing device sold by Leap Motion, Inc. having a place of business at San Francisco, Calif., USA. Generally, the LEAP MOTION® motion sensing device includes a first camera lens and a second camera lens that may image or view an object in a field of view of the LEAP MOTION® motion sensing device. The LEAP MOTION® motion sensing device can identify the portion to be tracked and identify movements of the tracked portion in space. For example, the instrument 30 can be positioned relative to the localizer 40, which may be the LEAP MOTION® motion sensing device, and the localizer can image the instrument 30 to track and/or identify the instrument and the navigation computer 70 can identify the instrument 30 in space and determine its position and movements in space. Software can be executed by the navigation computer 70 and include instructions embodied in code that is executed by the processor 70a, the software may be stored on the memory 70b.

The localizer device 40, may further include illuminating elements 46. The illuminating elements 46 may include infrared (IR) emitting elements. The emitting elements 46 may be appropriate elements, such as light emitting diodes (LEDs) or other appropriate emitting elements. The emitting elements 46 can insure proper illumination of the instrument 30, or other appropriate portion to be tracked during a selected procedure. The localizer 40 may include sensors, such as sensors of the cameras, which are sensitive to IR wavelengths or other appropriate wavelength.

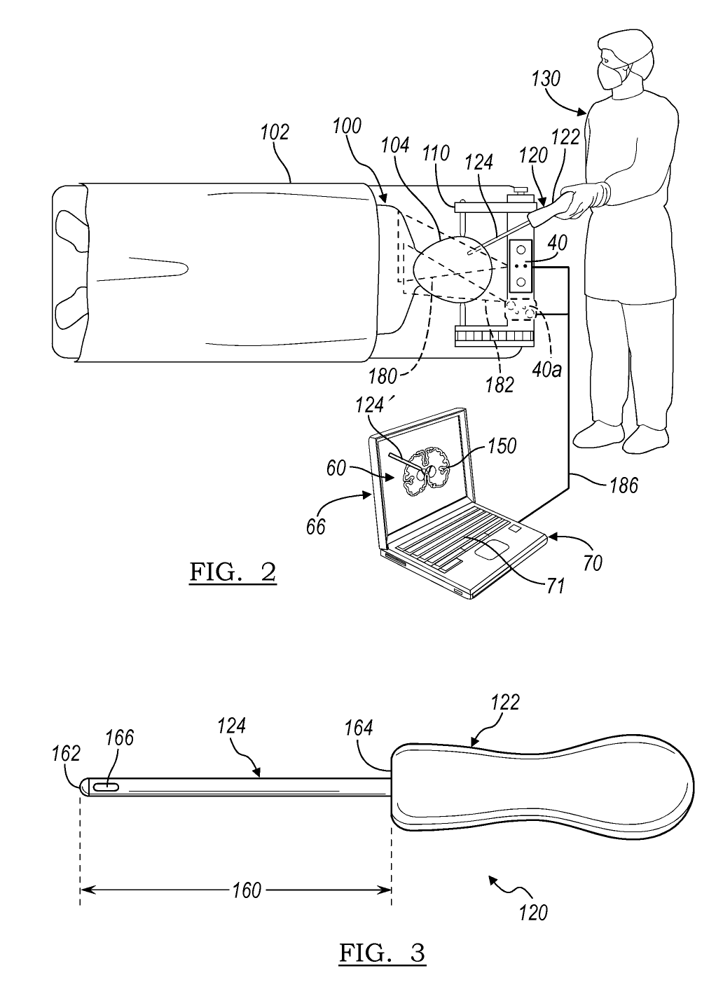

With reference to FIG. 2 and various embodiments, including a specific example, a procedure may occur on a human subject or object 100. The human subject or object 100 may be positioned in an operating room and on a table or support 102. Further, the patient or subject 100 may be held or partially held in the selected orientation. For example, a head 104 of the subject 100 may be held with a holder or fixation system 110. The holder 110 may include a MAYFIELD® skull clamp, such as those sold by Integra LifeSciences Corporation, that may be selectively attached to the head 104 to hold the head 104 in a selected position. Associated with the holder 110 may be the localizer 40.

The localizer 40 may be similar to that described above, including the LEAP MOTION® motion sensing device as discussed above. The localizer 40 can be used to track a position of a surgical instrument or intervention instrument 120. The intervention instrument 120, which may be a stylus, a pointer, a drill, an awl, etc. may include a manipulable portion or handle 122. The handle 122 may be held by a user, such as a physician 130. Further, the instrument 120 may include an intervention or operating end 124. The operating end 124 may be positioned within the head 104 of the subject 100 by the user 130. The instrument 120 can be used for various purposes such as biopsy (i.e., removal of selected material), shunt placement, deep-brain stimulation, or other appropriate procedures. It is further understood that non-brain procedures may also occur and the instrument 120 may be positioned relative to a selected portion of a patient, such as a spinal column for a spinal neural procedure, a chest cavity for a cardiac procedure, and other appropriate procedures. Nevertheless, the instrument 120 can be tracked with the localizer system 40 as discussed further herein. Also, the instrument 120 may be used to create gestures that are tracked with the localization system 40 for either performing the procedure and/or for interacting with the navigation system. Gestures may, for example, be used to change perspective of the imager, zoom of the image, opacity of the icon or rendering of the instrument 120, etc.

In the particular example, image data can be acquired of the subject 100 using various imaging techniques. For example, x-ray imaging, fluoroscopic imaging, magnetic resonance imaging (MRI), computer tomography (CT) imaging, and other appropriate imaging systems may be used to acquire or obtain the image data of the subject 100. The image data may be two-dimensional image data, three-dimensional image data, or two- or three-dimensional image data acquired over time to show change. For example, fluoroscopic or MRI image data can be acquired of the patient over time to illustrate motion of the various anatomical and physiological features, such as a heart rhythm. The image data can be saved and/or immediately transferred for display on a display device, such as the display device 66. The image 60 may include a direct image or a rendering based upon image data of a selected portion of the subject 100, such as a rendering or display of a brain 150.

An icon 124′ (which may be a representation of the instrument 120, which may further include a rendering of the instrument 120 including a three-dimensional rendering) can be used to illustrate a position of at least a selected portion, such as the intervention portion 124 of the instrument 120. The icon 124′ may be a three-dimensional rendering of the instrument 120 or selected portion thereof, such as only the intervention portion 124. The determination of the position of the intervention portion 124 can be made by tracking all or a portion of the instrument 120 and determining a location of the intervention portion 124, as discussed further herein. Therefore, the user 130 can view the display device 66 and the image 60 to understand the position of the intervention portion 124 by viewing the icon 124′ relative to the image of the brain 150, which is based upon the image data. As discussed above, registration of a position of the subject 100, including the head 104 relative to the localizer device 40, can be used to assist in appropriately illustrating the location of the icon 124′ relative to the brain rendering 150. Registration techniques can include those discussed above and others that are generally known in the art.

A location of the intervention portion 124 of the instrument 120 can be based upon known or input measurements of the instrument 120. With additional reference to FIG. 3, the instrument 120 is illustrated in greater detail. As noted above, the instrument 120 can include the handle 122 and the interventional portion 124. The handle 122 can be designed and provided in an appropriate configuration, such as an ergonomic shape including indentations and soft portions for efficient and proper grasping by the user 130. The handle 122 has an external surface contour or profile that may be imaged with the localizer 40. The external surface contour or profile can be recognized and tracked in the object space using the stereoscopic lens/camera of the localizer 40.

The intervention portion 124 can include a dimension 160 between a terminal distal end 162 of the intervention portion 124 and a distal terminal end 164 of the handle 122. The distance 160 can be used to determine the location of the distal terminal end 162 and other portions of the intervention portion 124, such as a portal or opening 166. The portal or opening 166 can include a portion that allows for resection or biopsy of a selected tissue. Further, the portal 166 may represent a stimulating region of an electrode or other operative portion.

Nevertheless, the localizer 40 may identify and track the handle 122 including the location of the distal terminal end 164 of the handle 122. Therefore, even if all or a portion of the intervention portion 124, including the distal terminal end 162, is not directly viewable or imageable by the localizer 40, a determination can be made, based upon the distance 160, of the position of the unviewed portion. Thus, the navigation computer 70 can determine the location of the portal 166 and the distal terminal end 162 and may display the icon 124′ at the position relative to the image 66 that represents the position of the intervention portion 124 relative to the subject 100.

The user 130 may enter into the system 70, such as with the user input portion 71, the distance 160. Alternatively, or in addition, the user may identify the instrument 120 and the system 70 may recall specific dimensions of the instrument 120, such as from the memory 70b. The memory 70b may include a database, such as a look-up table, of dimensions of specific instruments. Thus, the user 130 may identify the instrument, as discussed further herein. Also, the database may include an instrument's external surface contour. Thus, the external surface contour and the position of selected portions of the intervention portions relative to selected points, such as the distal terminal end 164 of the handle 122, may be saved in the database for tracking. Further, generally known techniques may be used to determine the location of a portion of the intervention portion 124 relative to the handle 122.

Accordingly, the localizer 40 can track the instrument 120, including a portion of the intervention portion 124, without a separate tracking member associated with the instrument 120. For example, a reflector portion or other tracking member need not be attached to the instrument 120. The instrument 120 may be a standard instrument that is not otherwise augmented or enhanced for use with a tracking system. The localizer 40 can be used to specifically identify a portion of the instrument 120, such as the handle 122, and track its position and the subject or object space so that the position of the instrument 120 can be determined as it is positioned relative to the subject 100.

A specific tracking member, such as a specifically positioned reflector or emitting device is not needed to be attached to the instrument 120 for tracking the instrument 120. This may allow for an efficient tracking of the instrument 120 during a procedure. Further, the tracking of the instrument 120 may occur without requiring additional attachments to the instrument, thus, the instrument 120 may be easily and efficiently used by the user 130 and the possibility of moving a tracking device relative to the instrument 120 is eliminated. Also, as no tracking member is required, no calibration of the position of the tracking member is required.

Further, as discussed above, and illustrated in FIG. 2, the localizer 40 may be provided as a single localizer element or portion. It is understood, however, as also illustrated in FIG. 2, that a second localizer 40a (illustrated in phantom), which is optional, may be provided. The second localizer 40a may be positioned near the first localizer 40 or may be positioned at another appropriate position. Exemplarily illustrated in FIG. 2 both the first localizer 40 and the second localizer 40a are positioned on the holder 110. Each of the respective localizers 40, 40a can have separate field of views 180, 182, respectively. The field of views 180, 180 may be co-extensive, such as to provide redundancy to the navigation system, may partially overlap to provide redundancy and a greater field of view, or may have entirely separate field of views such that an entire field of view of the tracking and navigation system may be increased relative to only a single of the localizers 40, 40a. Both of the localizers 40 and 40a can be connected to the navigation computer system 70 with one or more communication lines 186. The communication lines 186 can be wired, wireless, or other appropriate communications between the respective localizers 40 and 40a and the navigation computer 70. As discussed above, the tracking information can be sent from the localizer 40 and 40a to the navigation computer 70.

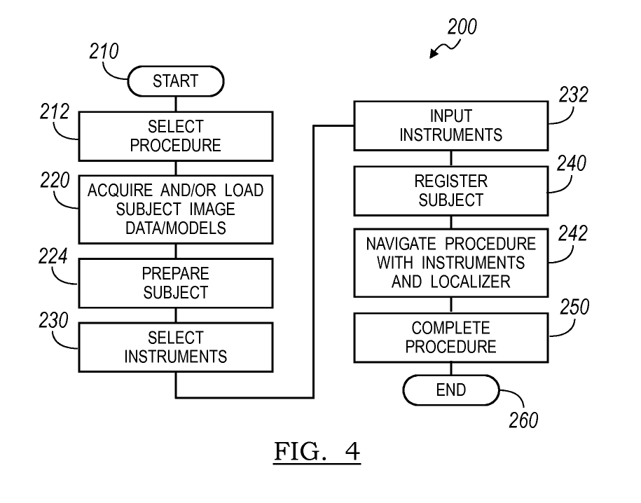

With additional reference to FIG. 4, a procedure 200 using the localizer 40 and/or 40a may be performed. Generally, the procedure may start in start block 210 and include selecting a procedure in block 212. Selecting a procedure can include selecting performing a biopsy, placing a stent, or other appropriate procedures. As noted above, various instruments can be used to perform selected procedures, such as biopsy of brain tissue or other tissues for placing of an implant such as a stent, catheter, vascular implant, or the like. The procedure can be selected to assist in determining the appropriate placement of the localizers, selection of an instrument or preparation of instruments and/or implants, and other procedural requirements.

As noted above, image 60 can be displayed on the display device 66. The image 60 may be a rendered model or may be raw image data that is displayed on the display device 66. Nevertheless, image data may be acquired and/or loaded in block 220. The acquisition of the image data can be performed with various imaging systems, including those discussed above, for example an MRI. The acquired image data may be stored in a patient storage system and may be loaded into the navigation computer 70 for the procedure 200. Additionally, models based upon the image data may be loaded or acquired for performing the procedure 200. The loaded image data or models may relate to the portion of the patient being operated on, including neurological models, heart models, or the like. The acquired image data may be displayed for illustrating the location of the instruments 120 relative to the subject 100 by displaying the instrument icon 124′ relative to the image 60 on the display device 66.

The subject may be prepared in block 224 at an appropriate time, such as before or after acquiring the image data or models of block 220. Preparation of the subject in block 224 may include fixing or holding the subject relative to the localizers 40, 40a such as with the holder 110. In exemplary embodiments, as discussed above, the holder 110 may be a Mayfield® skull clamp and may be used to fix the head 104 of the subject 100 in a selected location so that it is substantially immobile relative to the localizer 40. Preparation of the subject 100 may also include general surgical preparation such as cleaning, forming a burr hole, forming incisions, and other appropriate subject preparation.

Instruments may be selected in block 230 and the instruments or the selected instruments may then be identified or input into the navigation computer 70 in block 232. As discussed above, the instruments may include selected dimensions that are known relative to trackable or identifiable portions of the various instruments, including a selected external contour. As noted above, a distal end 164 of the handle 122 of the instrument 120 may be at the known distance 160 from the distal end 162 and/or or the portal 166 of the intervention portion 124. Therefore, selecting and inputting the instruments, in blocks 230, 232 respectively, may allow for the navigation computer 70 to identify position of various portions of the instrument 120 relative to other portions of the instrument 120.

As noted above, inputting the instruments in block 232 may include inputting specific dimensions of the instrument 120 for determining locations of portions thereof or other inputs, by the user 130. Further, inputting an identifying feature of the instrument 120 may allow the navigation computer 70 to load, such as from a database in the memory system 70b, predetermined dimensions of the instrument 120, including the dimension 160. The determination of the dimensions, however, may be performed in any appropriate manner. Also, the localizer 40 may image the instrument 120 to determine at least an external surface contour for tracking and at least the dimension 160. Thus, the localizer 40 may be used to determine dimensions for tracking in addition to or separate from any stored in a database. Also, this may allow for new or additional instruments to be used that are pre-identified and measured for storage in the database.

Once the subject is prepared in block 224, the subject may also be registered in block 240. Registration may proceed according to any appropriate registration technique, including those discussed above. Generally, registration allows for mapping of points in the subject space or object space to points in the image 60 or image space. Accordingly, registration may include identifying one or more points on the subject 100, such as on the head 104, and identifying the same points in the image 60 to allow for a mapping between the points or locations in the object space and points or locations in the image 60. Registration can allow for illustrating the instrument icon 124′ at the appropriate position relative to the image 60 based upon its tracked position relative to the subject 100, including the head 104.

During a procedure, registration may be maintained as the portion of the patient being acted upon on and relative to which the instrument 120 is being tracking is held fixed and substantially immobile (i.e. less than or equal to a tolerance of the system which may include a tracking accuracy) relative to the localization system 40. As illustrated in FIG. 2, the localization system 40 is fixed to the clamp 110 and the head 104 is fixed relative to the clamp 110. Thus, the head 104 is fixed relative to the localization system 40, and registration may be maintained. This may also reduce or eliminate the need of a patient tracking device to track the patient during a procedure to maintain registration.

Once registration occurs, navigation of the procedure with the instruments and the localizer may occur in block 242. Navigation may include illustrating the icon 124′ relative to the image 60 on the display device 66. Therefore, the user 130 may view on the display device 66 the position of the instrument 120 by viewing the icon 124′ representing the instant relative to the image 60. Thus, the user 130 may know the position of the instrument 120, including the operative end 124 relative to the subject 100 without directly viewing the operative end 124 within the subject 100.

The patient 100, including the head 104 or only the head 104 is generally held relative to the localizer 40, 40a with the holding device 110. Holding the head 104 relative to the localizer 40 maintains registration of the image space relative to the subject space. If the head 104, or other registered portion of the patient 100, moves relative to localizer 40 then the mapping of points between the patient 100 and the image 60 is no longer proper and navigation or illustration of the icon 124′ relative to the image is not correct. Thus, if the registered portion, such as the head 104, moves relative to the localizer 40 registration may need to occur again. Alternatively, a tracking member may be placed on the head 104, or other registered portion, to be tracked to maintain registration during a procedure. Further, the localizer 40 may be able to identify an external surface contour of the head 104, or other portion to be registered, and track the head during the procedure to maintain registration even with movement relative to the localizer 40.

The procedure may then be completed in block 250. Completion of the procedure may be obtaining the biopsy material, fixing a deep brain simulation probe, or completing other appropriate portions of a procedure. It may also include withdrawing an instrument and confirming that a selected procedure has been completed. The procedure may then end in block 260.

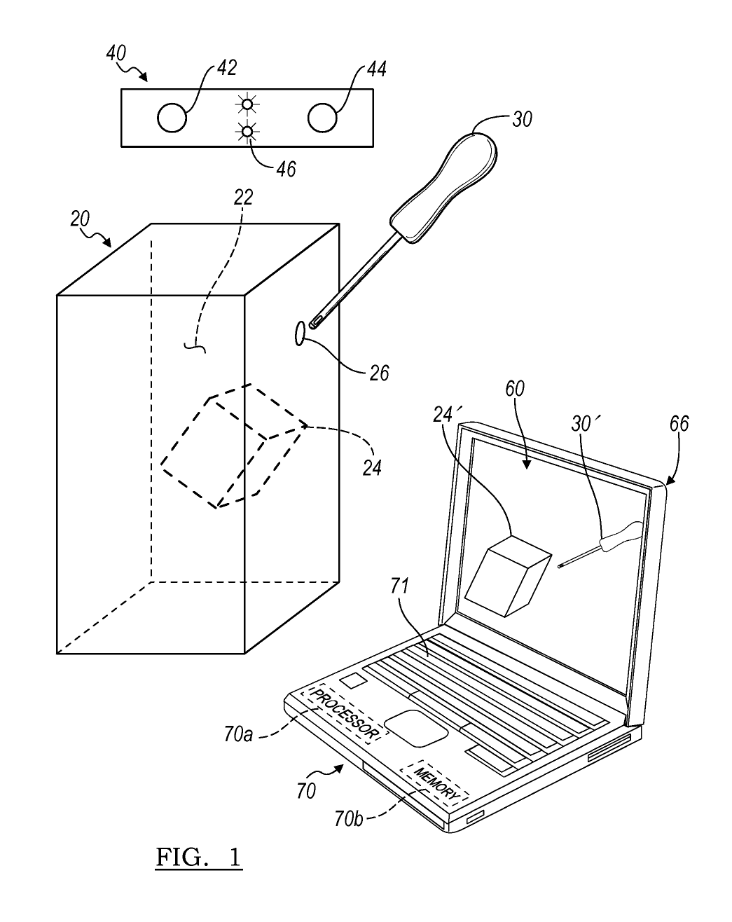

Therefore, the localizer, including the localizer 40 and/or 40a may be used to track and navigate the procedure relative to the subject 100. As noted above, the subject 100 need not be a human subject, and can be any appropriate object including that illustrated in FIG. 1. Nevertheless, the localizer 40 may be substantially small and efficiently positioned during a procedure. The localizer 40 may also be provided or supplied from general merchandise suppliers to allow for a cost-effective navigation system.

The instruments also may be provided in a manner that does not require a specific tracking member to be affixed thereto. Thus, the presently disclosed system allows for standard and unaltered instruments to be used to perform a navigated procedure, such as a navigated surgical procedure. Although the instruments may include inherent features (e.g., unique handle geometries), the inherent features may be substantially immovable relative to operative portions of the instrument. Therefore, placing and/or fixing tracking members at positions on the instrument, such as on the instrument 120, need not be considered. Therefore, navigating an instrument relative to an object, such as tracking the instrument 120 relative to the subject 100 and illustrating it via the icon 124′ relative to the image 60, is disclosed. The system allows for efficient and compact navigation of the instrument 120 relative to the subject, or other appropriate instruments relative to an object.

According to various embodiments, the localizer 40 may image all or a portion of the instrument 120. Once imaged, that portion may become the external surface contour that is tracked during a procedure. As discussed above, the localizer 40 includes lenses and or cameras that may image the instrument. Thus, the image of the instrument may be used to determine the external surface contour that is tracked during the procedure. For example, the external surface contour may include the transition point or contour from the handle 122 to the intervention portion 124 at the distal end 164 of the handle 122. This transition portion that defines the external surface contour that is used for tracking and allows the instrument 120 to be tracked without a separate tracking member attached to the instrument 120. Further, external surface contours may be pre-determined and saved in the database stores in the memory system 70b. Thus, when the user 130 inputs the instrument, such as in block 232, the navigation computer 70 may recall the external surface contour of the instrument for proper tracking thereof.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. Further, the various disclosed embodiments may be combined or portions of one example may be combined with another example in an appropriate manner. In some example embodiments, well-known processes, well-known device structures, and well-known technologies are not described in detail.

What is claimed is:

1. A navigation system, comprising:
an instrument having an external surface contour;
a localizer system configured to determine the external surface contour and determine a position of the instrument in space relative to an object based on the determined external profile; and
a display device configured to display an image of at least a portion of the object and an icon representing the determined position of the instrument relative to the image of at least the portion of the object.

2. The navigation system of claim 1, wherein the localizer system includes a first lens and a second lens;
wherein the first lens is positioned a distance from the second lens.

3. The navigation system of claim 2, wherein the localizer system further includes a processor system configured to determine the position of the instrument based on images acquired with at least one of the first lens or the second lens;
wherein the position includes at least six-degree of freedom position information.

4. The navigation system of claim 3, wherein the localizer system further includes the processor system configured to determine the position of the instrument based on images acquired with both of the first lens and the second lens.

5. The navigation system of claim 1, wherein the image of at least the portion of the object includes at least one of a magnetic resonance image or a computer tomography image.

6. The navigation system of claim 1, wherein the image of at least the portion of the object includes an image of a human subject.

7. The navigation system of claim 2, wherein the first lens includes a first camera and the second lens includes a second camera.

8. The navigation system of claim 2, further comprising:
a memory system including a database of information regarding a plurality of instruments, wherein the database includes dimensions of selected portions of each instrument of the plurality of instruments;
wherein the localizer system is configured to image at least one instrument of the plurality of instruments and the processor system is configured to execute instructions to determine a position of at least one selected portion of the selected portions of the instrument based on recalling the database of information.

9. A method of displaying an icon representing an instrument at a position of the instrument relative to an object in a navigation system, comprising:
determining an external surface contour of at least a portion of the instrument;
determining a position of the external surface contour of at least the portion of the instrument relative to the object with a placed first lens and a placed second lens relative to the object;
tracking movement of the external surface contour of at least the portion of the instrument relative to the object over time; and
displaying the icon with a display device based on the tracking movement.

10. The method of claim 9, further comprising:
displaying an image of the object on the display device; and
registering the displayed image of the object to the object.

11. The method of claim 10, further comprising:
superimposing the icon over the displayed image based on the determined position of the instrument.

12. The method of claim 9, wherein tracking movement of the external surface contour of the instrument includes tracking movement of only the external surface contour of the instrument.

13. The method of claim 9, further comprising:
acquiring the image of the object.

14. The method of claim 13, wherein acquiring the image of the object includes at least one of loading a magnetic resonance image or a computer tomography image.

15. A method of displaying an icon representing an instrument at a position of an instrument relative to an object in a navigation system, comprising:
fixing an object relative to a first localizer;
positioning the instrument within a first field of view of the first localizer;
operating the navigation system to:
determine a selected external surface contour of at least a portion of the instrument;
determine a position of the selected external surface contour of at least the portion of the instrument relative to the object with a placed first lens and a placed second lens relative to the object;
track movement of the external surface contour of at least the portion of the instrument relative to the object over time; and
display the icon with a display device based on the tracking movement.

16. The method of claim 15, further comprising:
placing a second localizer;
wherein the second localizer includes a third lens and a fourth lens;
wherein the second localizer is configured to at least one of duplicate the first field of view, generate a second field of view, or at least partially overlap the first field of view with the second field of view.

17. The method of claim 15, further comprising operating the navigation system to stereoscopically track the determined selected external surface contour of at least the portion of the instrument with at least the first lens and the second lens.

18. The method of claim 17, further comprising:
moving the instrument; and
operating the navigation system to illustrate the icon to represent movement of the instrument.

19. The method of claim 17, further comprising:
inputting an identification of the instrument; and
wherein operating the navigation system to determine the selected external surface contour of at least the portion of the instrument includes recalling from a database stored in a memory system the selected external surface contour of at least the portion of the instrument.

20. The method of claim 19, further comprising:
registering an object space relative to an image space of an image of the object;
maintaining the registration of the object space relative to the image space of the image of the object at least by the fixing the object relative to the first localizer.
